(19) United States
(12) Patent Application Publication
(10) Pub. No.: US 2013/0203057 A1
(43) Pub. Date: Aug. 8, 2013

(54) STEP-WISE DETECTION OF MULTIPLE TARGET SEQUENCES IN ISOTHERMAL NUCLEIC ACID AMPLIFICATION REACTIONS

(71) Applicant: BioHelix Corporation, Beverly, MA (US)

(72) Inventors: Bertrand Lemieux, Wenham, MA (US); Yanhong Tong, Boxford, MA (US); Hyun-Jin Kim, Beverly Farms, MA (US); Huimin Kong, Wenham, MA (US)

(73) Assignee: BIOHELIX CORPORATION, Beverly, MA (US)

(21) Appl. No.: 13/746,615

(22) Filed: Jan. 22, 2013

Related U.S. Application Data
(60) Provisional application No. 61/589,200, filed on Jan. 20, 2012.

(51) Int. Cl. C12Q 1/68

(52) U.S. Cl. CPC C12Q 1/6823
USPC 435/6.11

(57) ABSTRACT

Compositions and methods useful in nucleic acid assays are provided. The invention permits detection of multiple target sequences and control nucleic acids using isothermal nucleic acid amplification methods and subsequent detection of amplification products at different temperature steps by at least two probes with different annealing temperatures. This method can be used in isothermal nucleic acid amplification reactions to detect multiple targets of interest. In a particular example, cycling hybridization probes with different spectral and hybridization temperatures are used to detect different target sequences. Probes become fluorescent when they are cleaved by a thermostable ribonuclease, which only acts when the probes are hybridized to their respective templates.

### RELATED APPLICATIONS

This application claims the benefit of and priority to U.S. Provisional Application No. 61/589,200, filed Jan. 20, 2012, the entire contents of which are incorporated by reference herein.

### SEQUENCE LISTING

The instant application contains a Sequence Listing which has been submitted in ASCII format via EFS-Web and is hereby incorporated by reference in its entirety. Said ASCII copy, created on Jan. 24, 2013, is named BHX017.txt and is 1,867 bytes in size.

### FIELD OF INVENTION

This invention relates generally to the field of nucleic acid amplification chemistry. More specifically, it relates to the use multiplex isothermal nucleic acid amplification and detection.

### BACKGROUND

Thermophilic Helicase Dependent Amplification (tHDA) utilizes helicase to unwind double-stranded DNA to amplify nucleic acids without the need for temperature cycling used in the polymerase chain reaction. Currently tHDA employs the UvrD helicase from Thermoanaerobacter tengcongensis (Tte-UvrD helicase), and the large Klenow fragment of DNA polymerase I from Geobacillus stearothermophilus to achieve over a hundred million-fold amplification of a nucleic acid target sequence (An et al., J. Biol. Chem. 280, 28952-28958 (2005)). Thermostable single strand binding protein can also added to enhance performance. All these enzymes operate optimally in the 55° C. to 70° C. temperature range such that an isothermal incubation allows for the exponential amplification of deoxyribonucleic acids (DNA) when appropriate substrates are included in the reaction. Addition of a thermostable reverse transcriptase allows for the amplification of ribonucleic acids (RNA) when appropriate substrates are included in the reaction (Goldmeyer et al., J. Mol. Diagnostics. 9, 639-644 (2007)).

Like PCR, tHDA amplifies DNA or RNA using a single pair of synthetic oligonucleotide primers for each amplicon. These primers hybridize to the 5′ and 3′ edges of a target sequence and a DNA polymerase extends the primers through the addition of deoxynucleoside-triphosphates (dNTPs) to create double-stranded product. In PCR, the double-stranded products are separated through the use of heat, and the process is repeated to generate exponential amplification of the selected target. In tHDA, Tte-UvrD helicase separates the strands under isothermal incubation conditions. Adding a surplus of one of the 2 primers allows for the preferential formation of one of the two strands of DNA generated by tHDA (a.k.a. asymmetric isothermal amplification as described in U.S. Patent Publication No. 20110229887). Asymmetric PCR is a method well known in the art (e.g., U.S. Pat. No. 5,066,584; U.S. Pat. No. 6,309,833).

Asymmetric amplification allows for the use of nucleic acid hybridization to sequence-specific probes as a means of confirming the legitimacy of the amplification products as well as to provide a means of quality control in diagnostic tests through the detection of control templates that share the same primer binding sequence with a target sequence but differ in the probe binding sequence. The use of internal controls for real-time amplification is a method well known in the art (U.S. Pat. No. 6,312,929 and U.S. Pat. No. 7,728,123; U.S. Patent Publication Nos. 2005/0003374 A1 and 2006/0166232 A1). The aforementioned quality control is called competitive internal control (CIC), in that it utilizes a single pair of primers to simultaneously amplify both a target sequence of interest and a reference “internal control” sequence that can be amplified even when the target sequence is undetectable (J. Clin. Microbiol. 32:1354-1356 (1994)). When the target sequence and the CIC both fail to amplify, the test is declared as invalid and the likely causes of failure are 1) the presence of DNA amplification inhibitors in the sample or 2) loss of substrates or enzymes in the reaction that prevent amplification of DNA or RNA. In the case of tHDA, the efficiency of amplification of the target and its corresponding CIC are not necessarily equal such that when the target is in excess, the CIC may be out competed by the target since both share the same primer substrates (U.S. Patent Publication No. 20110229887). This unique particularity of tHDA has bearing on the present invention; a feature that does not apply in the case of PCR. Indeed, PCR benefits from pauses in the synthesis of DNA during the denature temperature part of the cycle that allow the slower reactions to catch up to the faster one while in tHDA DNA synthesis is continuous throughout the entire process.

Hybridized probes can be visualized through a variety of methods well known in the art that include the labeling of the probe with enzymes or luminescent or fluorescent reagents. In PCR, the detection of an amplified product with fluorescently labeled probes requires the use of specialized equipment such as fluorescence reading thermocyclers (a.k.a. real time thermocyclers). However, this specialized equipment is often costly.

The optics in the aforementioned instruments generally consist of a means of illuminating the sample within a specific range of wavelengths of electromagnetic radiation by passing the light through a cut-off filter, as well as a means of detecting the light emitted by a reporter group within a specific range of wavelengths of electromagnetic radiation after the reporter group has been excited. The wavelength of the emission is typically shifted towards the less energetic, red light end of the spectrum relative to the excitation maximum of a given dye.

A number of dyes are known in the art with non-overlapping emission spectra such that a properly configured instrument can typically monitor fluorescence increases in as many as 4 channels. Dyes that overlap in their excitation and emission spectra can undergo Förster resonance energy transfer (FRET) when they are appropriately spaced. Many fluorescence-based nucleic acid detection strategies rely on FRET using either 2 probes that are brought into proximity or single probes with 2 FRET partners that are cleaved between the 2 FRET partners to result in an increase in fluorescence of one of the partners.

Nucleic acids detection strategies depending on the hybridization of 2 single-labeled probes that can participate in a FRET (i.e., a donor and an acceptor probe) on adjacent segments of DNA or RNA are known in the art (U.S. Pat. No. 6,174,670, U.S. Pat. No. 6,245,514, U.S. Pat. No. 5,945,526). When combined with melting curve analysis of the hybridization probes, FRET allows for the detection of multiple target sequences (U.S. Pat. No. 6,140,054, U.S. Pat. No. 6,472,156). Unfortunately, melting curve analysis requires precise control of the rate of change in the incubation temperature of the reaction, such that the cost of the instrument used for such tests is far greater than that of instruments used for isothermal incubations.

Alternatively, when the dyes are on the same oligonucleotide, probes are said to be dual-labeled. The dual-labeled probes used in nucleic acid detection assays typically have two moieties; a reporter and quencher. These are typically spaced such as to allow the FRET transfer of the excited reporter to the quencher.

The mechanisms used to generate the fluorescence signal from a hybridized dual-labeled probe during DNA synthesis range from monitoring the release of a quenched dual-labeled probe using enzyme mediated probe degradation (U.S. Pat. No. 5,210,015; U.S. Pat. No. 7,122,364), to changes in probe conformation that alter the spacing between the reporter and quencher group to prevent FRET (Lukhtanov et al. 2007, U.S. Pat. No. 5,565,322; U.S. Pat. No. 7,015,018; U.S. Pat. No. 5,925,517; U.S. Pat. No. 6,103,476; U.S. Pat. No. 6,150,097; U.S. Pat. No. 7,385,043). Other methods that cleave the bond between a fluorescent reporter or a quencher and the probe using chemical means are also known in the art (U.S. Pat. No. 7,749,699; U.S. Pat. No. 7,745,614). When configured in the 2 adjacent probe format, these chemically uncaged fluorescent reporters allow for cyclical amplification of signal.

Some methods allow for the turnover of probes through physic-chemical or enzymatic mechanisms such that successive rounds of hybridization and signal generations are possible in isothermal conditions. The chemical turnover methods rely on the reduction of organic azides of individually designed profluorophores by triphenylphosphine (TPP) (U.S. Pat. No. 4,876,187; U.S. Pat. No. 5,011,769; U.S. Pat. No. 5,660,988; U.S. Pat. No. 7,968,289; U.S. Pat. No. 7,135,291). The application of this probe turnover technology to amplifying signal in nucleic acid amplification is restricted to amplification chemistries that operate at low temperatures because of the instability of the organic azide bonds and TPP in high temperature conditions. This vulnerability to temperature limits the utility of this approach in multiplex testing.

Among the enzymatic probe amplification strategies, one that is widely used is cycling probe technology (CPT) from ID Biomedical (U.S. Pat. No. 4,876,187; U.S. Pat. No. 5,011,769; U.S. Pat. No. 5,660,988; U.S. Pat. No. 6,503,709; U.S. Pat. No. 6,274,316; U.S. Pat. No. 7,122,314) and Takara Bio CataCleave™ probes (U.S. Pat. No. 7,135,291; U.S. Pat. No. 6,951,722; U.S. Pat. No. 7,056,671; U.S. Pat. No. 7,135,291; Harvey et al., Anal Biochem. 333(2):246-55 (2004); Harvey et al., J Clin Lab Anal. 22(3):192-203 (2008) Mukai et al., J Biochem. 142(2):273-81 (2007)). CPT uses a chimeric DNA-RNA-DNA probe containing a plurality of ribonucleotides that hybridizes to a complementary target DNA sequence (a.k.a., chimeric DNA-(RNA)₂₋ₓ-DNA probes). The enzyme RNAse H recognizes the resulting hybrid and cleaves the RNA portion of the duplex, resulting in cleavage. The shorter probe fragments dissociate from the complex because they are less stable than the starting probe thus freeing the target sequence to bind another DNA-RNA-DNA probe. Solid phase versions of this detection chemistry have been invented (e.g., U.S. Pat. No. 6,596,489 U.S. Patent Publication No. 20110269129).

A novel variant of this method is use of dual-labeled probes and a thermostable RNAse HII, which allows such a reaction to use a single centrally located ribonucleotide (DNA-(RNA)₁-DNA probes) to hybridize to a complementary target sequence (Liu et al., Anal Biochem. 398(1):83-92 (2010); Hou et al., Oligonucleotides. 17(4):433-43 (2007)). This variant method is distinct for the CPT and CataCleave technologies, which use a plurality of RNA residues to detect specific sequences using RNAse H. By exploiting the specificity of cleavage of the thermostable RNase HII for perfectly matched duplexes at the single RNA base, the assay is less dependent on binding as a means of detection. Indeed, technologies dependent on chimeric DNA-(RNA)₂₋ₓ-DNA probes get their sequence-specificity by introducing additional purposeful mismatches relative to a given target when designing their probes. Purposeful mismatches prevent binding of the probe to the mismatched target such that the RNAse HI cannot cleave the DNA-(RNA)₂₋ₓ-DNA probe. This design is necessary because any of the RNAs in the DNA-(RNA)₂₋ₓ-DNA probe can be cut, thus probes with a single mismatch relative to their targets might still be cut even when the single nucleotide difference is at one of the several RNA moieties.

### SUMMARY OF THE INVENTION

The methods and compositions described herein offer advantages over nucleic acid detection strategies known in the art. For example, use of tHDA to amplify a target sequence allows for use of lower cost optical detection instruments than those required for PCR (e.g., real time thermocyclers). In addition, the detection strategies described herein allow for isothermal incubation conditions and therefore do not require the more expensive instruments required for melting curve detection, which precisely control the rate of temperature. The use of lower cost instruments is a significant advantage for the wider acceptance of nucleic acid amplification technology and represents a significant advantage of this invention.

In one aspect, a method is provided for detecting target nucleic acids. The methods includes the steps of (a) performing an isothermal nucleic acid amplification reaction to amplify at least a first target nucleic acid and a second target nucleic acid; (b) detecting the first target nucleic acid with a first probe at a first temperature; and (c) detecting the second target nucleic acid with a second probe at a second, different temperature, such that the first probe is able to bind the first target sequence at the first temperature and not at the second temperature. In certain embodiments, the first probe is cleaved by a nuclease. In some embodiments, the nuclease is RNase HII, e.g., a thermostable RNase HII. In one embodiment, the isothermal nucleic acid amplification is a Helicase-Dependent Amplification. In certain embodiments, each detecting step is performed at a substantially constant temperature.

In another embodiments, kits for detecting target nucleic acids are provided. A kit can include, e.g., at least two amplification primers for performing an isothermal nucleic acid amplification reaction to amplify at least a first target nucleic acid and a second target nucleic acid, a first probe capable of detecting the first target nucleic acid at a first temperature, and a second probe capable of detecting the second target nucleic acid at a second, different temperature, wherein the first and second probes are cleavable by RNase HII when bound to their respective targets. In some embodiments, the isothermal nucleic acid amplification is a Helicase-Dependent Amplification. In other embodiments, the kit further comprises RNase HII, e.g., a thermostable RNase HII. In some embodiments, the kit further comprises a helicase.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 depicts the activity of the RNAse HII at different temperatures. A fluorescein-labeled DNA-(RNA)₁-DNA probe was hybridized to a template DNA with a quencher to generate a substrate DNA. Typically, 5 pg of the substrate DNA was incubated at 40° C., 50° C., 65° C., and 75° C., and fluorescence increase was measured in an ABI 7300 temperature-controlled fluorimeter.

FIGS. 2A and 2B depict multiplex schemes for detecting competitive internal control templates after the target nucleic acid was not detected. FIG. 2A shows the detection strategy for up to 6 targets using incubation at 65° C. to detect the targets in real time. In this example, all possible 6 targets are present and detected during the incubation and thus, there is no need to perform the optional incubation at 50° C. to detect the corresponding CIC templates. FIG. 2B shows the detection strategy for up to 6 targets. In this case only 2 targets are detected during the incubation at 65° C. The optional incubation at 50° C. is used to detect the CIC templates corresponding to the targets that were not detected at 65° C. to confirm the amplification reagents used to detect these 4 missing targets are operating correctly.

FIGS. 3a and 3b depict detection of HSV-1 and HSV-2 along with the HSV competitive internal control template. Real-time detection HSV-1 vs. HSV-2 on Tube Scanner occurred by implementing CPT together with HDA. Fluorescence monitoring in real-time is shown as fluorescence intensity versus time. FIG. 3a shows the FAM view for HSV-2 detection. FIG. 3b shows the TAMRA view for HSV-1 detection. HSV2 low (50 copies/reaction of HSV-2 DNA), HSV1 high (5000 copies/reaction of HSV-1 DNA), HSV1 low (50 copies/reaction of HSV-1 DNA) and NTC (non-template control) were tested in duplicate.

FIG. 4 depicts detection of CIC after amplification. In the case of HSV-1 and HSV-2 negative samples, after incubation at 64° C. for 45 minutes, the reaction is cooled to 35° C. to allow the CIC probe to bind to the amplicons generated from the CIC template. An initial fluorescence intensity reading is done after 1 minute at 35° C. and the temperature is increased to 50° C. to allow for cyclical cleavage of the CIC probe. During this 50° C., measurements of fluorescence each minute shows a linear increase in fluorescence, indicating that additional probes bind to the template and are cleaved. By reducing the temperature to 35° C. after 10 minutes at 50° C., a greater number of probes can bind to the template. Returning to 50° C. and measuring fluorescence each minute for 5 minutes shows a linear increase in fluorescence.

### DETAILED DESCRIPTION OF EMBODIMENTS

The compositions and methods described herein relate to detection of multiple target sequences using isothermal nucleic acid amplification methods and subsequent detection of amplification products at different temperature steps by at least two probes with different annealing temperatures. These methods can be used in isothermal nucleic acid amplification reactions to detect multiple targets of interest. In one embodiment, thermostable RNAse HII, DNA-(RNA)₁-DNA probes of different lengths can be used in combination with multiple incubation temperatures.

Probes suitable for use with the methods described herein typically have a reporter group and a quencher. The fluorescence emission maxima of these reporters are typically spaced at least 40 nM apart such as to allow for an independent detection each probe using commercial fluorescence detection system. Specific embodiments include probe sets consisting of 6-carboxy fluorescein (6-FAM) (Abmax 495 nm Emmax 520 nm), or MAX 557 (Abmax 524 nm Emmax 557 nm), with TYE 563 (Abmax 549 nm Emmax 563 nm), TEX 615 (Abmax 596 nm Emmax 613 nm), TYE 665 (Abmax 645 nm Emmax 665 nm), and TYE705 (Abmax 686 nm Emmax 704) such that up to 6 different fluorescence detection channels are possible when a properly configured instrument is used to perform the assays. Preferred embodiments use low-cost fluorimeters with 2 or 3 detection channels; generally 6-FAM & TYE 563 or 6-FAM & TEX 615 or 6-FAM & TYE665 or 6-FAM & TYE705 or 6-FAM & TYE 563 & TYE665 or TYE705 or 6-FAM & TEX 615 & TYE665 or TYE705.

The methods described herein increase the level of multiplexing of nucleic acid detection assays performed in instruments that lack the capability to perform of smooth temperature ramps needed for melting curve analysis based multiplexing by using probe cleavage to generate fluorescence increase. In certain embodiments of the present invention, each detecting step is performed at a substantially constant temperature. For example, a detection reaction may be held at the temperature required for binding of a first probe to its target nucleic acid for anywhere from 1 second to several hours. In some embodiments, a detection reaction is held at the temperature required for binding of a first probe to its target nucleic acid for at least 1 second, at least 2 seconds, at least 5 seconds, at least 30 seconds, or at least one minute.

Another advantage of the methods described herein is that the activity of the thermostable RNAse HII is adequate over a sufficiently large range of temperatures such as to allow for probes with different melting temperatures to bind in order to control the cleavage of the probes. As these probes are cleaved, the products of the digestion are less stable on the template DNA, and thus dissociate from the template, allowing a new intact probe to hybridize under the isothermal incubation conditions.

When these DNA-(RNA)₁-DNA probes are used in combination with an isothermal nucleic acid amplification like HDA, probes with a 64° C.-66° C. melting temperature are detected during amplification. Using a lower annealing temperature after the amplification reaction allows for a distinct post-amplification detection of a second set of probes using a brief isothermal incubation during which shorter probes can be detected. Specific embodiments use probes with a 40° C.-60° C. melting temperatures. Preferred embodiments use probes with 45° C.-55° C. melting temperatures. This innovation allows to at least double the number of target sequences that can be detected with a given set of fluorescent dual labeled probes.

When the lower melting temperature DNA-(RNA)₁-DNA probes are probes for detecting CIC templates, this invention allows for the conditional detection of the CIC in multiplex diagnostic assays performed on negative samples. In such embodiments, a target sequence can be detected with a probe with a melting temperature of 64° C.-66° C. in each of the 6 possible fluorescence detection channels, and their respective CIC templates are detected by CIC specific probes with 40° C.-60° C. melting temperatures using the same fluorescence reporter dye as the their corresponding target sequences. As such this invention allows for at least 12-plex detection when 6 dyes are used in the homogeneous probe detection format. As a 10° C. separation between the melting temperatures of probes is generally sufficient to provide adequate discrimination in probe hybridization, a multiplex detection level of 18-plex is possible when 6 dyes are used in the homogeneous probe detection format.

### EXAMPLES

### Example 1

### Determination of the Activity of Thermostable RNAse HII at Different Incubation Temperatures

A double stranded template was generated using the oligonucleotides: H2-T1: 5′-CGC CTC CCA TCT CCT GCA TCA CCT CAC GAG-BHQ_{—}1-3′ (SEQ ID NO:1) and H2-P1: 5′-FAM-CTC GTG AG rG TGA TGC AGG AGA TGG GAG GCG-3′ (SEQ ID NO:2; where “rG” is the ribonucleotide moiety in the sequence). The 2 oligonucleotides were mixed in 1:1 molar ratio, incubated at 95° C. for 10 minutes, and cooled down to room temperature to form double stranded DNA substrates for the thermostable RNAse HII activity assays.

Assays for estimating the specific activity of RNAseHII used 50 μL reactions containing:
120 nM dsDNA substrate
5 μL Rnase HII (serial dilutions)
1×ROX
20 mM Tris-HCl
10 mM (NH4)2SO4
10 mM KCl
2 mM MgSO4
0.1% Triton X-100
pH 8.8 at 25° C.

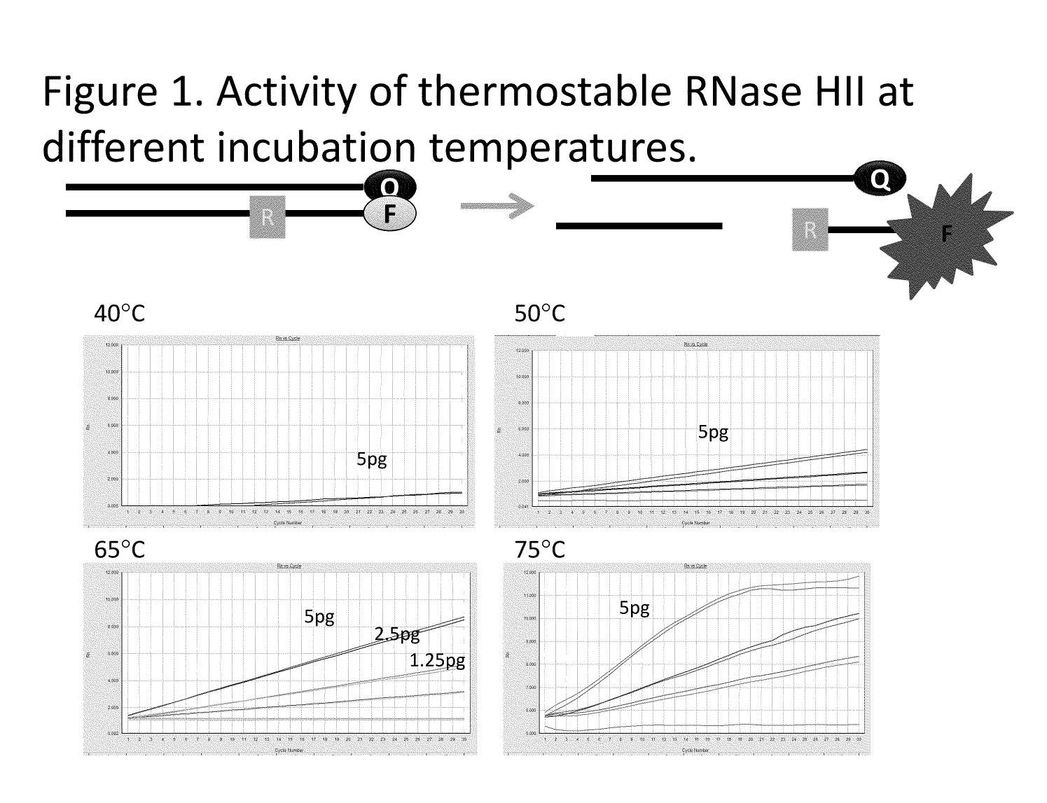

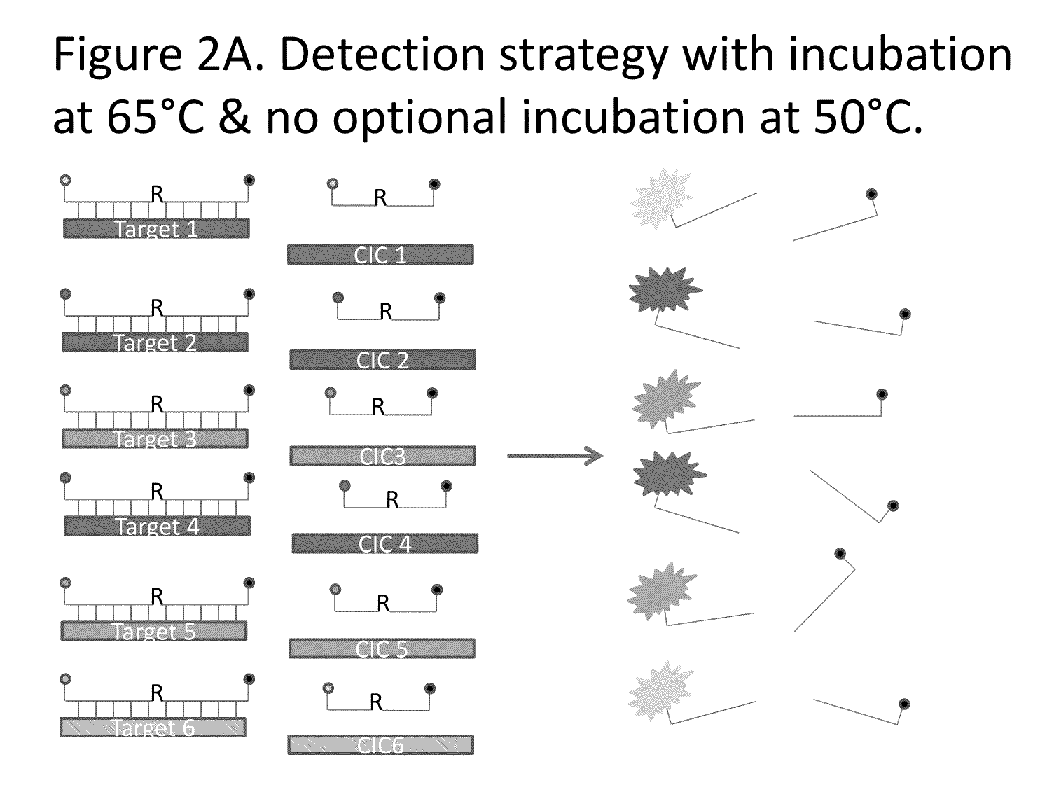

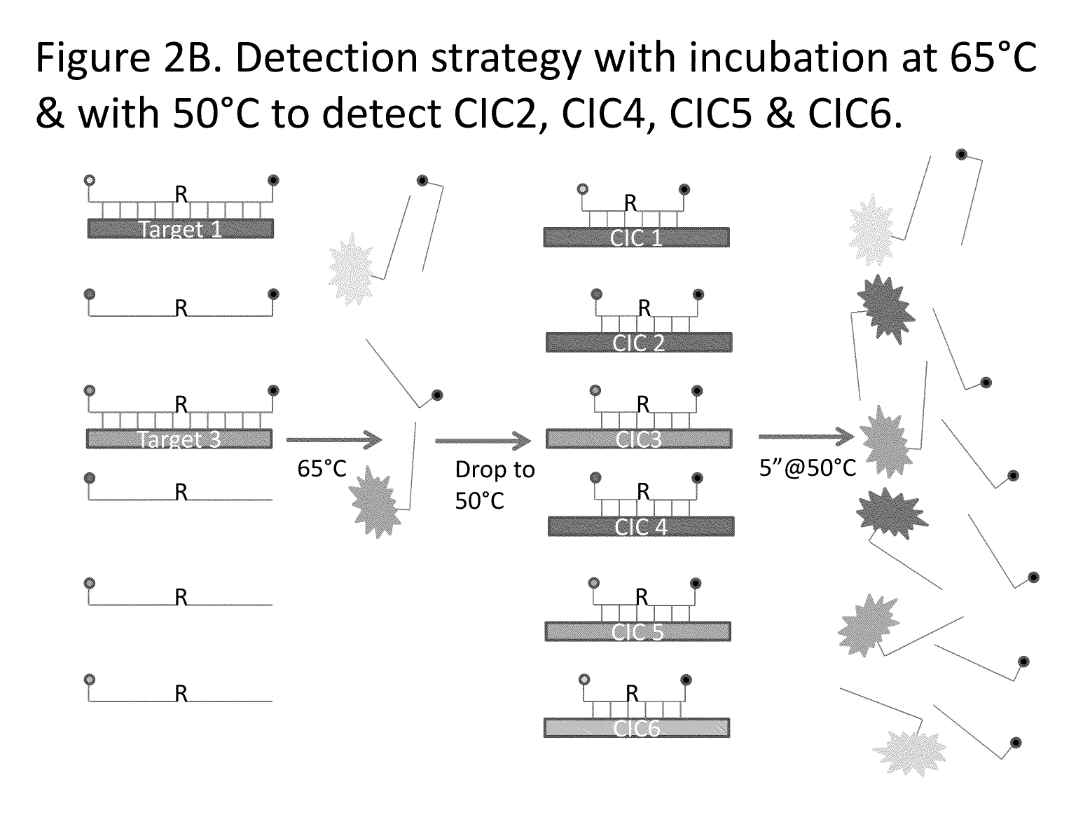

The reactions were incubated at 40° C., 50° C., 65° C., or 75° C. for 30 minutes and fluorescence signals were read every minute. One unit is defined as the amount of enzyme required to yield a fluorescence signal consistent with the nicking of 100 picomoles of synthetic double-stranded DNA substrate containing a single ribonucleotide in 30 minutes at 65° C. FIG. 1 shows the data obtained using a ABI 7300 fluorimeter while incubating the reactions at 40° C., 50° C., 65° C., or 75° C. for 30 minutes. Activity of the thermostable RNAse HII is highest at 75° C., but significant activity remains at 40° C.

### Example 2

### Detection of HSV-1 and HSV-2 along with the HSV Competitive Internal Control Template

The primers for the amplification of HSV are identical to those reported in Kim et al. Journal of Clinical Virology 50(1): 26-30 (2010). The probes for the IsoGlow™ HSV typing assay are listed in Table 1. These probes were designed with a Tm around 65° C., therefore, allowing them to bind to the corresponding complementary sequence efficiently at 64° C., and to be cleaved by RNAse HII during the amplification of HSV DNA. Because of the presence of two polymorphisms between HSV-1 and HSV-2 in the probe binding regions (underlined in Table 1), these probes are subtype-specific. As the probes are labeled with different reporter groups, each HSV type can be detected using a different fluorescence channel. The HSV CIC probe used to detect the HSV CIC is also listed in Table 1. The HSV CIC has the same primer binding sequence as HSV-1 and HSV-2 such that it is amplified by the primers reported in Kim et al. Journal of Clinical Virology 50(1): 26-30 (2010). The DNA sequence of the HSV CIC between the two primers is entirely different from that of HSV-1 and HSV-2 and the melting temperature of the HSV CIC probe is 50° C. As the activity of the thermostable RNAse HII is high at a wide range of temperatures (FIG. 1), sufficient HSV CIC probe is cleaved to generate fluorescence signal after as little as 1 minute to yield detectable signal.

TABLE 1
IsoGlow ™ HSV typing probes

| Probes | Sequences and modifications |
|---|---|
| HS-1 typing probe | 5′-TYE563-CGTCACCGTTTcGCAGGTGTG-3BHQ-1 (SEQ ID NO: 3) |
| HS-2 typing probe | 5′-FAM-CGTGACcGTGTCGCAGG-3BHQ-1 (SEQ ID NO: 4) |
| HSV CIC probe | 5′-TYE563-TACGAAGGCGAcAAA-3′BHQ-1 (SEQ ID NO: 5) |

Quantified HSV-1 or HSV-2 viral DNA from Advanced Biotechnologies Inc. (Columbia, Md.) were used as a gold standard. The concentrations of HSV-1 or HSV-2 positive plasmid from IsoAmp HSV were determined and verified by qPCR using the gold standard and the same primer pairs used for HDA.

The qPCR was performed with DyNAmo™ HS SYBR® Green qPCR Kit with 400 nM of each primer per reaction (25 μL/reaction). The qPCR assay was performed at ABI 7300 with the following program: 15 minutes of 95° C., and then 40 cycles of 10 seconds at 94° C., 30 seconds at 55° C., 30 seconds at 72° C. (with fluoresce data collection), 3 minutes of 72° C. for final extension followed by melting curve analysis. The IsoGlow™ HSV typing assay was initially optimized with HSV-1 and HSV-2 plasmid, and finally validated with quantified HSV-1 or HSV-2 viral DNA.

The viral transport mediums were collected from Lab Alliance of Central New York (NY). The samples were shipped on ice for overnight delivery, and were aliquoted upon receipt. Some were placed at −80° C. for long term storage, and some were placed at −20° C. for short-term storage and immediate testing.

The IsoGlow™ HSV typing enzyme reagent was prepared, similar to that of IsoAmp HSV kit as described before, with the modification of using 2×IsoAmp III enzyme mix plus RNase HII enzymes (a recombinant protein purified from E. coli cloned the gene encoding the thermostable RNase HII, at a concentration of 20 ng per assay). The IsoGlow™ HSV typing amplification reagent for platform one was prepared as follows: for each assay, 60 nM of HSV-1 typing probe, 60 nM of HSV-2 typing probe, and 60 nM of HSV IC probe 1, were combined with the other components in the IsoAmp HSV amplification reagent (primers and buffers). The IsoGlow™ HSV typing amplification reagent for platform two was prepared similar to that of platform one, with the HSV IC probe 1 replaced by 80 nM of HSV CIC probe 2. The internal control was premixed in the amplification reagent for both platforms.

The IsoGlow™ HSV typing assays were done using the following set-up: the reaction master mix was prepared by combining 40 μL IsoGlow™ HSV amplification reagents, and 5 μL IsoGlow™ HSV enzyme reagent per assay. Five μL of viral transport medium were mixed together with HSV dilution buffer. Five μL of the tested sample was mixed together with the 45 μL master mix in 200 μL thin-wall PCR tubes (Bio-Rad, Hercules, Calif.), and covered with mineral oil to prevent cross contamination. The tubes were placed in ESE-Quant Tube Scanner for amplification and detection. The tubes were placed in ESE-Quant Tube Scanner for amplification and detection. The statistical analysis of the results was performed by the “Data analysis” program using Excel software.

Tube Scanner using the following program:
35° C. for 1 minute with data collection every 20 seconds (for initial-point data collection, three data points, N35 is the average of the three readings at 35° C.),
64° C. for 45 minutes with data collection every 1 minute (monitor real-time amplification, N64 is the average of the first four readings at 64° C., S64 is the average of the last four readings at 64° C.),
35° C. for 1 minutes without data collection (for IC probe maximally annealing to the target),
46° C. for 10 minutes with data collection every 30 seconds (for IC probe cleavage, the data collection is optional for this step),
35° C. for 2 minutes with data collection every 30 seconds (for end-point data collection, four data points, S35 is the average of the four readings at 35° C.).

The final results determined by detecting the signal to noise in the Tamra channel at 64° C. (T_{(S64/N64)}), the signal to noise in the fluorescein channel (F_{(S35/N35)}) and the signal to noise in Tamra channel at 35° C. T_{(S35/N35)}) for HSV-1, HSV-2, and the CIC, respectively.

TABLE 2
Performance of two formats of IsoGlow ™ HSV assay on 60 clinical
samples in comparison to ELVIS ® shell vial assay.

| | | Reference method | | | |
|---|---|---|---|---|---|
| | | HSV-1+ | HSV-2+ | HSV− | Total |
| IsoGlow ™ | HSV-1+ | 20 | NA | 0 | 20 |
| HSV typing | HSV-2+ | NA | 20 | 0 | 20 |
| Using cut-offs | HSV− | 0^{a} | 0 | 20 | 20 |
| In Table 3 | Total | 20 | 20 | 20 | 60 |

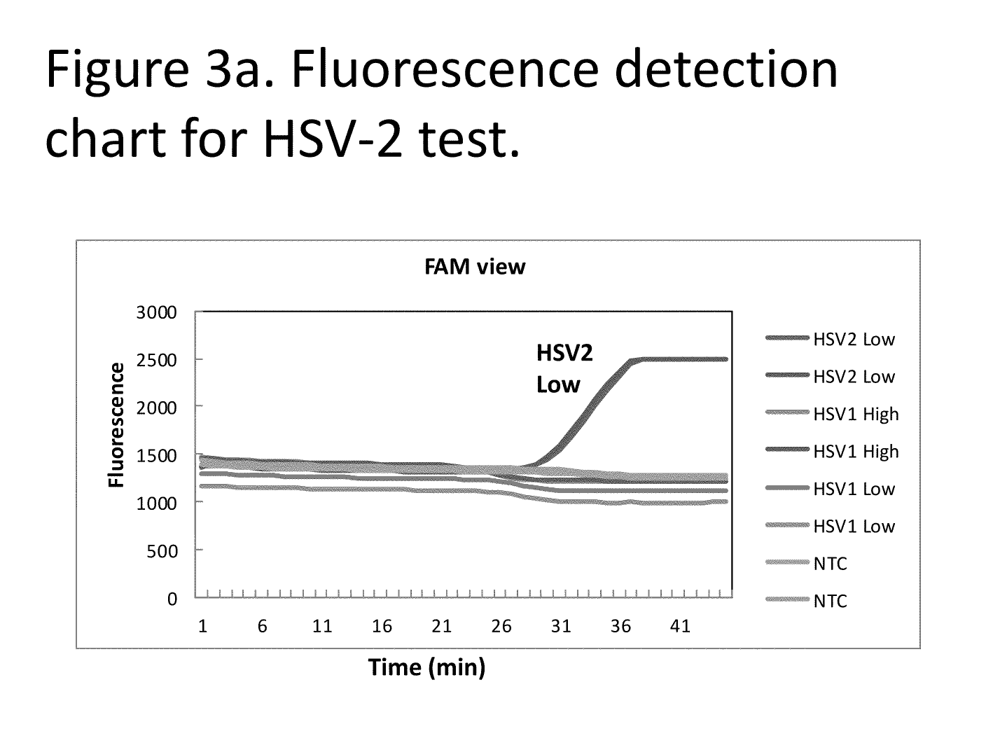

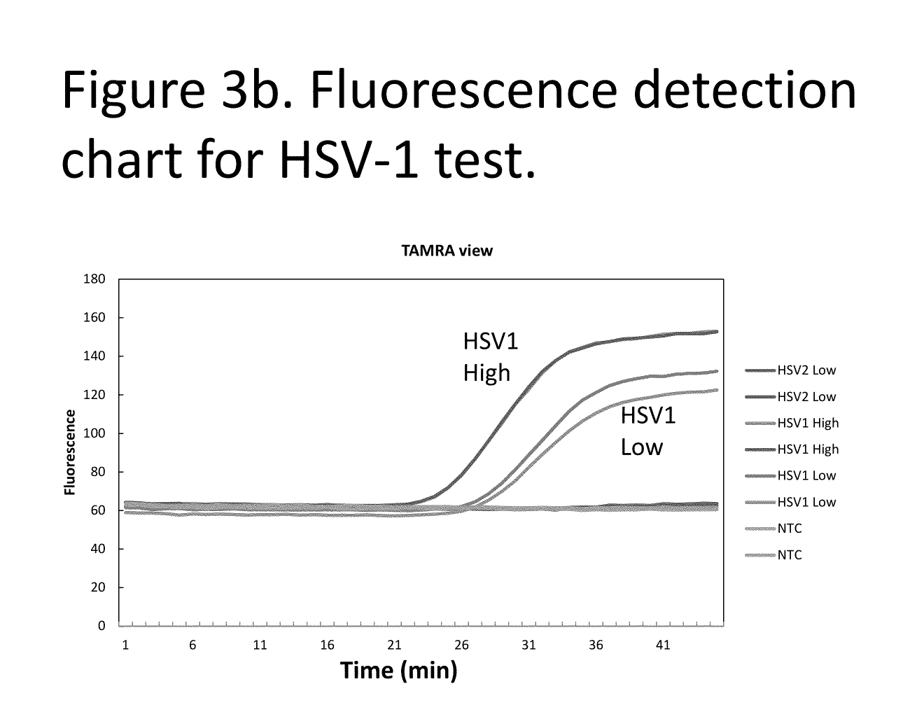

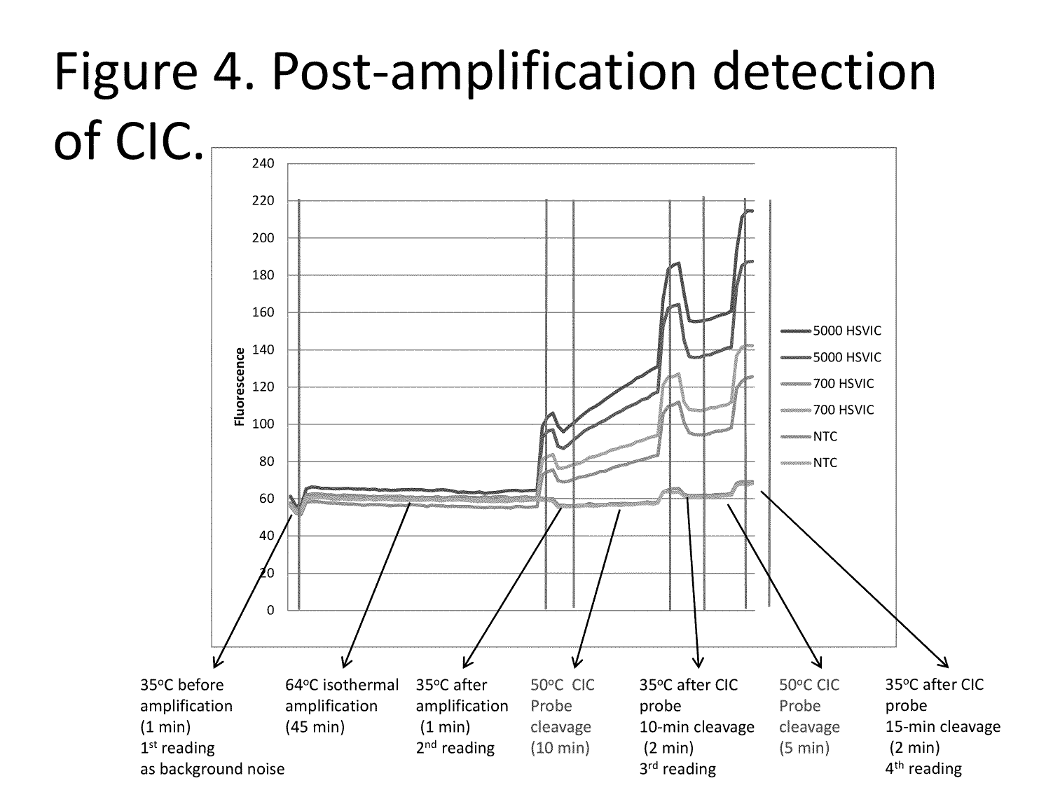

The fluorescence increase observed during the incubation at 64° C. is used to detect the HSV-1 and HSV-2 target templates. FIGS. 3A and 3B are examples of the fluorescence increase detected during the amplification of the templates. The 64° C. incubation is only followed by incubations at lower temperatures in the case of HSV-1 and HSV-2 negative samples; i.e., when such incubations are needed to distinguish between true negative results and invalid results using the CIC template amplification as a reporter. When such lower temperature incubations are needed, we have observed that a lower temperature touch down at 35° C. (i.e., 15° C. below the Tm of the CIC probe) increases the number of probes bound to the template. We have also observed the linear increase in fluorescence during incubations at 50° C. As this invention pertains to the end-point detection in fluorescence increase to detect the presence or absence of the target and CIC templates, the increase in fluorescence is obtained by setting a minimum threshold for the ratio of the absolute fluorescence at the beginning of a step and a set point determined by the test manufacturer.

TABLE 3
Fluorescence ratio thresholds observed with clinical
samples and empirically determined cut-offs

| | | | |
|---|---|---|---|
| Detector | Range | Mean | Standard Deviation |

19 HSV1 positive samples, T (S₆₄/N₆₄) cutoff = 1.5

| | | | |
|---|---|---|---|
| TAMRA (64° C.) | 1.6-2.6 | 2.4 | 0.2 |
| FAM (35° C.) | 1.1-1.2 | 1.1 | 0.0 |

20 HSV2 positive samples, F (S₃₅/N₃₅) cutoff = 2.5

| | | | |
|---|---|---|---|
| TAMRA (64° C.) | 1.0-1.4 | 1.1 | 0.1 |
| FAM (35° C.) | 6.6-7.8 | 7.1 | 0.3 |

21 HSV negative samples, T (S₃₅/N₃₅) cutoff = 2

| | | | |
|---|---|---|---|
| TAMRA (64° C.) | 1.0-1.2 | 1.0 | 0.1 |
| TAMRA (35° C.) | 2.8-4.5 | 3.4 | 0.4 |
| FAM (35° C.) | 1.1-1.5 | 1.2 | 0.1 |

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

What is claimed is:

1. A method for detecting target nucleic acids, the method comprising:
a) performing an isothermal nucleic acid amplification reaction to amplify at least a first target nucleic acid and a second target nucleic acid;
b) detecting the first target nucleic acid with a first probe at a first temperature; and
c) detecting the second target nucleic acid with a second probe at a second, different temperature, wherein the first probe is able to bind the first target sequence at the first temperature and not at the second temperature.

2. A method according to claim 1, wherein the first probe is cleaved by a nuclease.

3. A method according to claim 2, wherein the nuclease is RNase HII.

4. A method according to claim 3, wherein the RNase HII is a thermostable enzyme.

5. A method according to claim 1, wherein the isothermal nucleic acid amplification is a Helicase-Dependent Amplification.

6. A method according to claim 1, wherein each of steps b and c are performed at a substantially constant temperature.

7. A kit for detecting target nucleic acids, the kit comprising:
a) at least two amplification primers for performing an isothermal nucleic acid amplification reaction to amplify at least a first target nucleic acid and a second target nucleic acid;
b) a first probe capable of detecting the first target nucleic acid at a first temperature; and
c) a second probe capable of detecting the second target nucleic acid at a second, different temperature, wherein the first and second probes are cleavable by RNase HII when bound to their respective targets.

8. A kit according to claim 6, wherein the isothermal nucleic acid amplification is a Helicase-Dependent Amplification.

9. A kit according to claim 6, wherein the kit further comprises RNase HII.

10. A kit according to claim 6, wherein the kit further comprises a helicase.


SEQUENCE LISTING (5 sequences)

SEQ ID NO: 1 - DNA, 30 bp, Artificial Sequence
  Description of Artificial Sequence Synthetic oligonucleotide
cgcctcccatctcctgcatcacctcacgag

SEQ ID NO: 2 - DNA, 30 bp, Artificial Sequence
  Description of Artificial Sequence Synthetic oligonucleotide
ctcgtgaggtgatgcaggagatgggaggcg

SEQ ID NO: 3 - DNA, 21 bp, Artificial Sequence
  Description of Artificial Sequence Synthetic probe
cgtcaccgtttcgcaggtgtg

SEQ ID NO: 4 - DNA, 17 bp, Artificial Sequence
  Description of Artificial Sequence Synthetic probe
cgtgaccgtgtcgcagg

SEQ ID NO: 5 - DNA, 15 bp, Artificial Sequence
  Description of Artificial Sequence Synthetic probe
tacgaaggcgacaaa